(19) United States
(12) Patent Application Publication
(10) Pub. No.: US 2017/0333027 A1
(43) Pub. Date: Nov. 23, 2017

(54) BONE ANCHOR ASSEMBLY

(71) Applicant: RAMBAM MED-TECH LTD., Haifa (IL)

(72) Inventors: Yaniv Keren, Kibutz Gesher Aziv (IL); Yaron Berkovich, Kibutz Ein Carmel (IL)

(21) Appl. No.: 15/526,769

(22) PCT Filed: Nov. 12, 2015
(86) PCT No.: PCT/IB2015/058752

§ 371 Date: May 15, 2017

Related U.S. Application Data
(60) Provisional application No. 62/079,174, filed on Nov. 13, 2014.

(51) Int. Cl. A61B 17/04; A61B 17/56

(52) U.S. Cl. CPC A61B 17/0401; A61B 17/56; A61B 2017/0411; A61B 2017/0446; A61B 2017/044

(57) ABSTRACT

Embodiments of the bone anchor assembly (36, 50) described herein have adjustable lengths and therefore accommodate many needs. Therefore, the physician does not need to maintain a multitude of bone anchors having a variety of lengths in order to be prepared for many situations. The bone anchor assembly (36, 50) has an elongated hollow shaft portion (38, 52) formed of multiple shaft segments (46, 58) joined together. The length of the shaft portion (38, 52) is adjusted after the implanting of the bone anchor assembly into the bone by removing shaft segments (46, 58).

### RELATED APPLICATION

This application claims benefit under 35 U.S.C. §119(e) of the Nov. 13, 2014 filing of U.S. Provisional Application No. 62/079,174, which is hereby incorporated by reference in its entirety.

### BACKGROUND

Bone anchors, sometimes referred to as “suture anchors,” lodge into a bone of a patient and hold one or more sutures securely thereto, and the sutures in turn securely hold soft tissue, such as tendons and ligaments. Accordingly, bone anchors are useful for many types of both open and arthroscopic orthopedic surgery, for example, for rotator cuff repair.

An exemplary conventional bone anchor 10 is illustrated in FIG. 1. This bone anchor 10 has exterior threads 12, like an ordinary wood screw, and a post 14 on top having an eyelet 16 for a suture 18 to be threaded therethrough. (In other exemplary conventional bone anchors, sutures are embedded within the anchor material instead of remaining easily slidable through an eyelet.) A physician implants the bone anchor 10 using an orthopedic drill, such as one manufactured by Striker, DePuy Synthes Companies of Johnson and Johnson, or Zimmer Biomet.

Care must be exercised that the bone anchor 10, while being lodged sufficiently deeply into the bone to secure it thereto, is not lodged so deep that the suture 18 is susceptible to contacting the bone and perhaps weakening the suture 18. Weakening of the suture 18 may occur due to chemical processes such as hydrolytic and enzymatic degradation caused by bone contact, and these chemical processes can eventually dissolve the suture material. (Although the dissolving of the suture may be desired eventually, it is not desired early in the patient's healing process.) Also, the drilling of the bone may have roughened the bones' surface where it could contact the suture, and a jagged surface may wear down the suture prematurely.

FIG. 2 provides an illustration of bone anchors 20 and 22 already implanted in a patient's bone 24, so as a result only the top heads of the bone anchors 20 and 22 are visible in the figure. Sutures 26, 28, 30, and 32 join the bone anchors 20 and 22 to the patient's soft tissue 34. As discussed above, it is undesirable to submerge the bone anchors 20 and 22 so far into the bone 24 that the sutures 26, 28, 30, and 32 contact the interior of the drilled hole (not shown). As one can see from FIG. 2, it is also undesirable that the heads of the bone anchors 20 and 22 would protrude too far above the surface of the bone 24. Accordingly, the physician needs bone anchors having a length that is long enough for reaching depths as low as necessary for solid anchorage into the particular bone and the part of that bone being drilled, but bone anchors cannot be so long as to protrude from the bone surface.

Due to the wide variety of particular bones into which bone anchors may be lodged, the different optimum depths for particular bone areas require a variety of lengths of bone anchors to be available to the physician. That is, for a given bone having a given optimum depth, a bone anchor must have a certain length so that it is not too short and not too long. It can however become burdensome to maintain a supply of bone anchors of different lengths for all the anticipated needs a physician may have.

As is clear, the purpose of using a longer length for a bone anchor is to effect a more secure lodging. Another way though to effect a more secure lodging while keeping the anchorage shallow is to increase the bone anchor diameter. However, using bone anchors of larger diameters decreases the number of individual bone anchors that may be implanted into a given area of bone. Thus, the stress from the sutured soft tissue is distributed among fewer bone anchors, thus increasing the stress on each individual bone anchor and on the associated sutures in use. Also, after some treatments, the shallow anchorage can lead to the loss of the fixation of the bone anchors, anyway, even if the bone anchor diameters are greater.

The present inventors decided to develop a bone anchor that was suitable to implantation in bones at a variety of depths according to the particular needs, so thereby (1) not being susceptible to the disadvantages of requiring large diameters that lessen the number of bone anchors that could be implanted, (2) not requiring maintaining a multitude of different bone anchor lengths, and (3) easily supporting sutures in a fashion to avoid contact with the surrounding bone.

### SUMMARY

The present inventors have developed a bone anchor assembly that adapts to many applications, because it is adjusted to a suitable length after it has been implanted. Alternate embodiments have different ways to adjust the length of the bone anchor assembly.

The invention may be embodied as a bone anchor assembly for implanting into a bone. The bone anchor assembly has: an elongated hollow shaft portion; a threaded end portion; and sutures. The elongated hollow shaft portion is formed of multiple shaft segments joined together at shaft segment boundaries. The threaded end portion is joined to the shaft portion. The sutures are joined to the end portion and extend inside the shaft portion. The length of the shaft portion may be adjusted after the implanting of the bone anchor assembly into the bone by removing shaft segments.

The bone anchor assembly may further have a shaft axis along which the elongated hollow shaft portion extends. For this implementation, shaft segments may be removed by applying a force to the shaft portion in a direction normal to the shaft axis and sufficient to deform the shaft portion to cause the shaft portion to break at one of the shaft segment boundaries between two adjacent shaft segments.

Alternatively, the bone anchor assembly may further be configured such that the shaft segments have tabs at one axial end and blanks at the opposite axial end so that the shaft segments interlock by mating the tabs of one shaft segment with the blanks of an adjacent shaft segment.

Embodiments of the present invention are described in detail below with reference to the accompanying drawings, which are briefly described as follows:

### BRIEF DESCRIPTION OF THE DRAWINGS

The invention is described below in the appended claims, which are read in view of the accompanying description including the following drawings, wherein:

FIG. 1 illustrates a conventional bone anchor;

FIG. 2 illustrates conventional bone anchor, such as that of FIG. 1, implanted into a patient's bone and attached to a patient's soft tissue by sutures;

FIG. 3 provides a perspective view of a bone anchor assembly in accordance with a first embodiment of the invention;

FIG. 4A provides a side view of a bone anchor assembly in accordance with a second embodiment of the invention;

FIG. 4B provides a cross-sectional view of the bone anchor assembly of FIG. 4A;

FIG. 4C provides a cross-sectional view of the bone anchor assembly of FIG. 4A at the A-A cut-away designated in FIG. 4B viewing in the direction of the arrows;

FIG. 4D provides a cross-sectional view of the bone anchor assembly of FIG. 4A at the B-B cut-away designated in FIG. 4B viewing in the direction of the arrows;

FIG. 4E provides a perspective view of two engaged shaft segments of the bone anchor assembly of FIG. 4A; and

FIG. 4F illustrates the bone anchor assembly of FIG. 4A in use after implantation.

### DETAILED DESCRIPTION

The invention summarized above and defined by the claims below will be better understood by referring to the present detailed description of embodiments of the invention. This description is not intended to limit the scope of claims but instead to provide examples of the invention. As disclosed herein, the present bone anchor assembly may be implanted into bone at a variety of depths. For embodiments of the bone anchor assembly, the length is set after it is lodged into the bone. That is, it is a one-size that fits many applications, because it is adjusted to the particular depth after the implantation procedure begins.

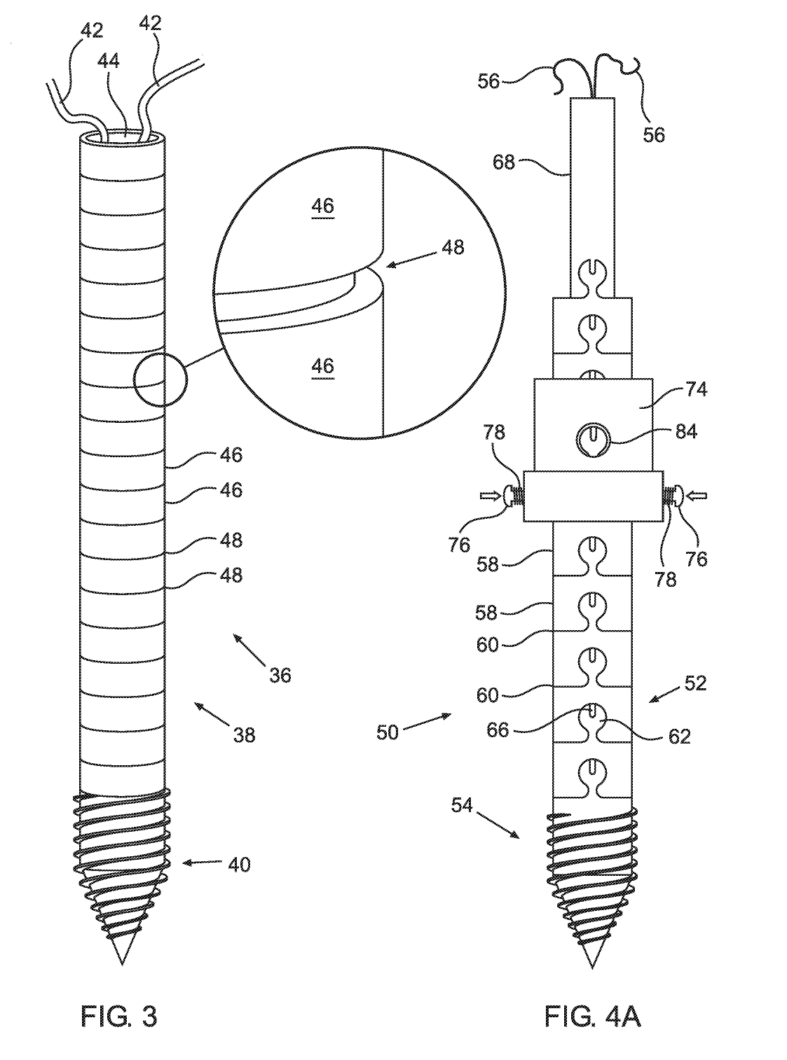

An exemplary embodiment of the invention is illustrated in FIG. 3. As shown, a bone anchor assembly 36 for implanting into a bone has an elongated hollow shaft portion 38, a threaded end portion 40, and sutures 42. (Throughout the disclosure, the term “sutures” may be used to refer to single strand of thread with two segments for tying together, or the term may refer to multiple threads, as non-limiting examples.) The end portion 40 of the bone anchor assembly 36 is joined to the shaft portion 38, for example, by welding or by forming the shaft portion 38 and the end portion 40 as a unitary piece. The bone anchor assembly 36 of the present embodiment has two sutures 42, but the invention is not limited accordingly.

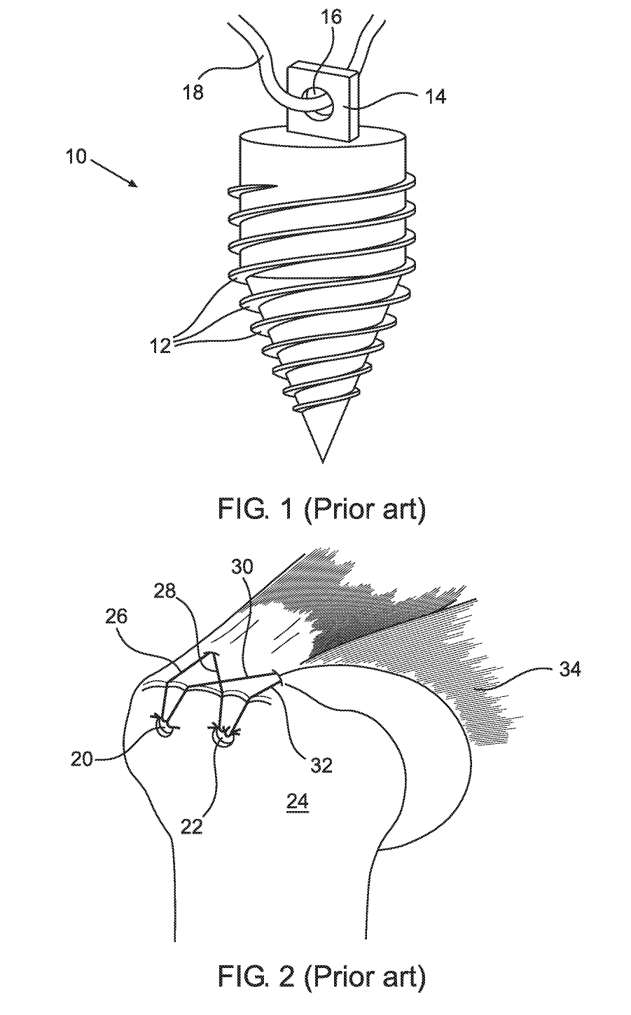

Conventional sutures may be used in this embodiment of the bone anchor assembly 36. The sutures 42 are joined to the end portion 40 of the bone anchor assembly 36 in any manner determined by one skilled in the art using conventional considerations and technology. For example, the sutures 42 may be joined to the end portion 40 of the bone anchor assembly 36 in the fashion that the suture 18 in FIG. 1 is joined to the prior art bone anchor 10. The sutures 42 of the bone anchor assembly 36 extend inside the shaft portion 38 along the shaft portion axis to exit the bone anchor assembly 36 at an opening 44 in the shaft portion 38 at an end distal to the end portion 40. If desired, the sutures 42 may be sized long enough to extend along the shaft axis and beyond the shaft portion 38 itself.

The shaft portion 38 is formed of multiple shaft segments 46, and the shaft segments 46 are joined together at shaft segment boundaries 48. With reference also to the circled enlarged section of the shaft portion 38 in FIG. 3, it is clear that the shaft segments 46 have external cylindrical walls that have a given diameter, and the shaft segment boundaries 48 have external cylindrical walls that have another diameter that is less than diameter of the external walls of the shaft segments 46. Such is intentional to make the cylindrical wall of the shaft portion 38 weaker at the shaft segment boundaries 48 than at the shaft segments 46. One non-limiting way to decrease the diameter of the exterior wall of the shaft portion 38 at the shaft segment boundaries 48 to implement laser cutting to remove wall material where desired. Another way to decrease diameter of the exterior wall of the shaft portion 38 is to rotate the bone anchor assembly on a lathe and mechanically remove material where desired. Reducing wall diameter is one way to weaken the wall of the shaft portion 38 at the shaft segment boundaries 48, but other ways of weakening the wall are within the scope of the present invention.

The material used to manufacture the shaft portion 38 and the end portion 40 may be determined by those skilled in the art according to needs and available resources. Non-limiting examples of such materials include continuous carbon fibers reinforced polymer, biodegradable materials such as PLDLA (Poly-L-co-D.L-lactic), and metal, such as such as titanium and titanium alloys.

The length of the shaft portion 38 of the bone anchor assembly 36 may be adjusted after the implanting the bone anchor assembly into a patient's bone by removing the shaft segments 46 that extend beyond the surface of the bone. One way to remove those shaft segments 46 is to apply a force to the shaft portion 38 in a direction normal to the shaft axis and sufficient to deform the shaft portion 38 to cause the shaft portion 38 to break at one of the shaft segment boundaries 48 between two adjacent shaft segments 46. As it is desirable to break off and remove all shaft segments 46 that extend beyond the bone after the bone anchor assembly 36 is implanted, the shaft portion 38 should break at the shaft segment boundary 48 between the submerged shaft segment 46 that is closest to the bone surface and the adjacent shaft segment that protrudes from the bone. Thus, while applying the force to the shaft portion 38 normal to the shaft axis, the force should be focused so that it is stronger at the shaft segment boundary 48 to be broken than at another shaft segment boundary 48.

One way to focus the shaft-bending force on the shaft segment boundary 48 to be broken is to slide a collar, for example, a long tube, outside and down the shaft segment 46 the bone surface. The collar may be designed to be long enough so that, is use, all shaft segment boundaries are surrounded by either the bone or the collar, except for the shaft segment boundary 48 to be broken.

After the shaft portion 38 is broken at the desired shaft segment boundary 48, one broken part of the shaft portion 38 is surrounded by bone, and the other broken part is completely external to the bone. The latter part may be removed and discarded. The sutures 42 may be joined to soft tissue to complete the care for the patient.

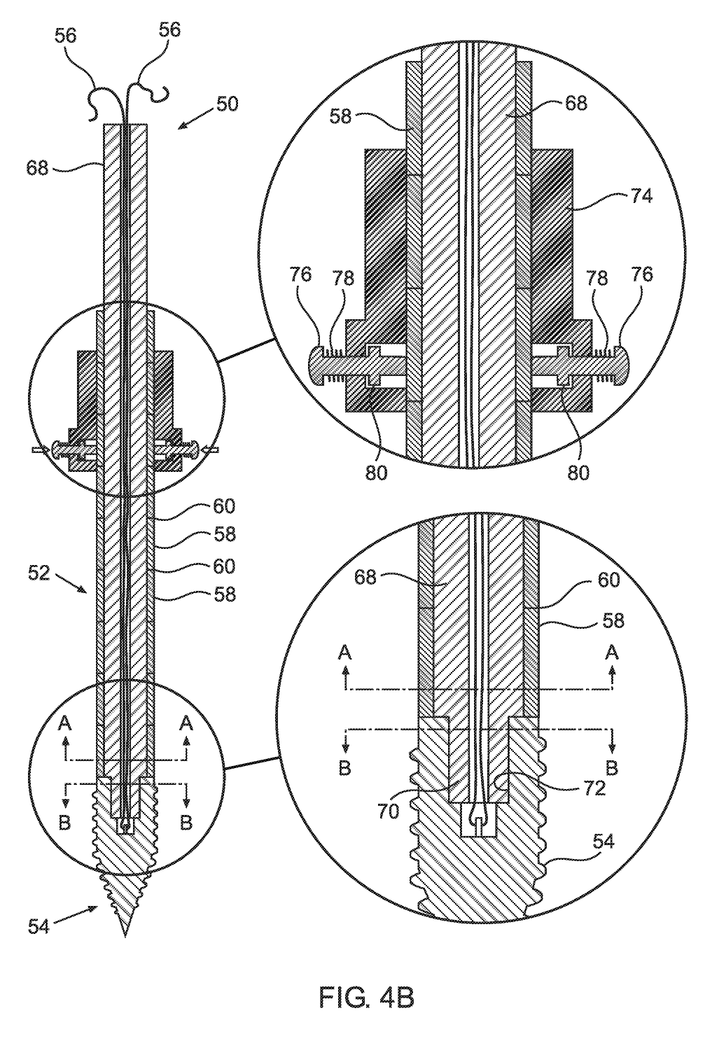

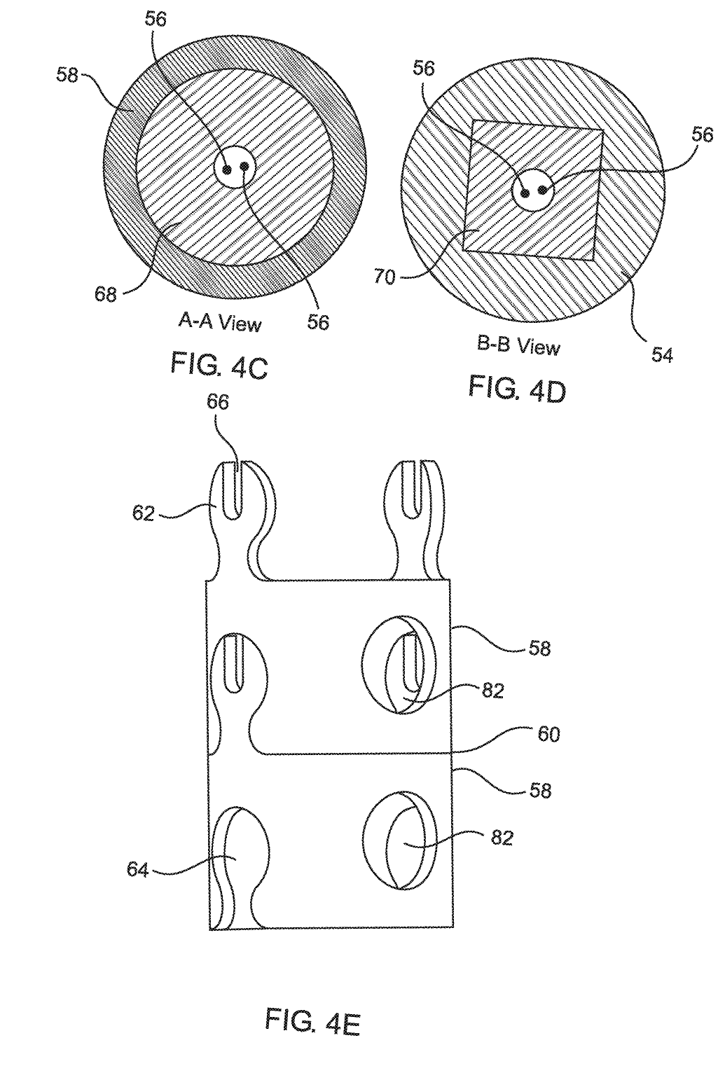

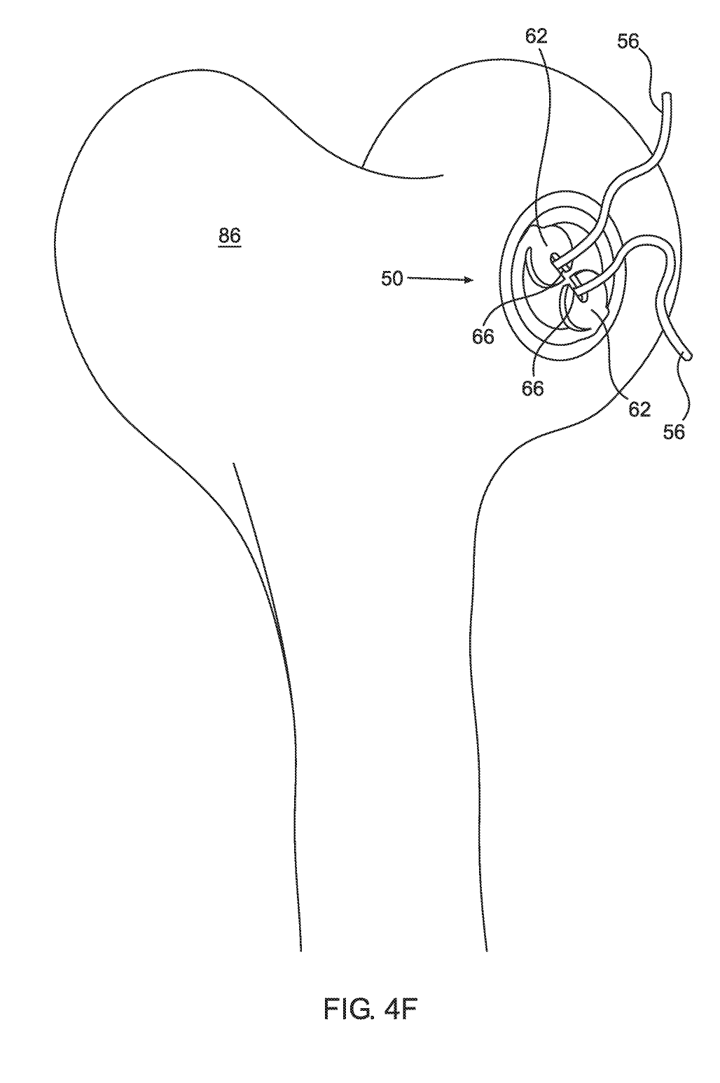

An alternate exemplary embodiment of the invention is illustrated in FIGS. 4A-4F. As shown, a bone anchor assembly 50 for implanting into a bone has an elongated hollow shaft portion 52, a threaded end portion 54, and sutures 56. The end portion 54 of the bone anchor assembly 50 is joined to the shaft portion 52, for example, by welding or by forming the shaft portion 52 and the end portion 54 as a unitary piece. The bone anchor assembly 50 of this embodiment has two sutures 56.

As in the embodiment of FIG. 3, conventional sutures may be used in this embodiment, also. The sutures 56 are joined to the end portion 54 of the bone anchor assembly 50 in any manner determined by one skilled in the art using conventional considerations and technology. The sutures 56 of the bone anchor assembly 50 extend inside the shaft portion 52 along the shaft portion axis to exit the bone anchor assembly 50 at an end distal to the end portion 54. If desired, the sutures 56 may be sized long enough to extend along the shaft axis and beyond the shaft portion 52 itself. The shaft portion 52 is formed of multiple shaft segments 58, and the shaft segments 58 join together at shaft segment boundaries 60.

With reference to FIGS. 4A and 4E, it can be seen that the shaft segment boundaries 60 resemble boundaries between jigsaw puzzle pieces. More specifically, a given shaft segments 58 has tabs 62 at one axial end and blanks 64 at the opposite axial end so that the shaft segments 58 interlock by mating the tabs 62 of one shaft segment 58 with the blanks 64 of an adjacent shaft segment 58. Optionally, the tabs 62 may be formed having grooves 66 extending in the axial direction, the grooves 66 functioning to position the sutures 56 away from the bone as discussed below.

To rotate the end portion 54 of the bone anchor assembly 50, a hollow driving rod 68, sized and shaped to fit into the shaft portion 52, is inserted therein to engage the end portion 54. Thus, the driving rod 68 may be coupled to a conventional orthopedic drill, and rotating the driving rod 68 causes rotation of the end portion 54 to drill the bone anchor assembly 50 into the bone of a patient. Upon such engagement of the driving rod 68 with the end portion 54, the sutures 56 extend inside the driving rod 68 along the shaft axis.

With reference to FIG. 4C, along most of the length of the driving rod 68, the driving rod 68 it has a circular cross section. However, at the end of the driving rod 68 proximal to the end portion 54 the driving rod 68 has an engagement portion 70 with a non-circular cross-section. See FIG. 4D, which shows that the engagement portion 70 as a square-shaped cross section. (Other shapes, such as that of a hexagon, may be used in other implementations.) The engagement portion 70 of the driving rod mates (engages) with a correspondingly shaped socket 72 in the end portion 54.

As with the embodiment of FIG. 3, in the present embodiment the length of the shaft portion 52 is adjusted after the implanting the bone anchor assembly 50 into a bone by removing shaft segments 58. To remove shaft segments 58 in this embodiment, first the driving rod 68 is withdrawn from the interior of the shaft portion 52 and end portion 54. Then, a collar 74 surrounding the shaft portion 52 is slid thereon toward the end portion 54 until the collar 74 approaches the bone surface. The collar 74 is then aligned with a shaft segment 58 as discussed next and separates the shaft segment 58 from an adjacent shaft segment 58. Note that the collar 74 positioned as illustrated in FIG. 4A needs to be rotated ninety degrees around the shaft axis in order to separate shaft segments 58. The collar is nonetheless illustrated as shown to provide a better view of its elements.

The collar 74 has sliding pistons 76 as radially-inward moving elements. As will be explained, the pistons 76 slide in the direction of the arrows in FIGS. 4A and 4B to separate shaft segments 58. The pistons 76 are biased by coil springs 78 so as not to contact the shaft segments 58. Larger diameter sections 80 of the pistons 76 prevent the pistons 76 from ejecting from the collar 74. In alternate embodiments, the coil springs 78 may be replaced with leaf springs joined to a collar, with the leaf spring having protrusions extending inwardly in place of the pistons 76 of the present embodiment.

Regarding the present embodiment, when the collar 74 is aligned along the shaft axis properly with respect to shaft segment 58 that is to be disengaged from an adjacent shaft segment 58, forcing the pistons 76 to slide against their biasings toward the shaft segment 58 moves the tabs 62 out of engagement with the blanks 64 of the adjacent shaft segment 58. In some implementations of the present embodiment, to effect the proper alignment of the collar 74 with the shaft segment 58 to be disengaged, the shaft segments 58 have radial holes 82 and the collar 74 has at least one radial hole 84. A physician can see through the hole 84 in the collar 74 when the hole 84 is aligned with a hole 82 of a shaft segment 58.

The holes 82 in the shaft segments 58 have the added benefits of reducing the amount of metal in the bone and allowing bone “in-growth” to improve bone-implant integration and stability. However, because the presence of the holes 82, it is desired to position the sutures 56 close to the shaft axis to avoid contact with the bone. Accordingly, reference is made to FIG. 4F illustrating the bone anchor assembly 50 after its implantation into a bone 86 and after the shaft segments 58 that were not submerged into the bone 86 are removed. The tabs 62 are bent ninety degrees from their original position, and the sutures 56 extend from the bone anchor assembly 50 through the grooves 66 in the tabs 62, thus keeping the sutures 56 farther from the part of the bone 86 that was drilled.

Having thus described exemplary embodiments of the invention, it will be apparent that various alterations, modifications, and improvements will readily occur to those skilled in the art. Alternations, modifications, and improvements of the disclosed invention, although not expressly described above, are nonetheless intended and implied to be within spirit and scope of the invention. For example, the disclosed collar may be modified so that it does not completely surround a shaft portion, thereby have a “U-shaped” as opposed to an “O-shaped” cross-section. Accordingly, the foregoing discussion is intended to be illustrative only; the invention is limited and defined only by the following claims and equivalents thereto.

1. A bone anchor assembly for implanting into a bone, the bone anchor assembly comprising:
an elongated hollow shaft portion formed of multiple shaft segments joined together at shaft segment boundaries;
a threaded end portion joined to the shaft portion; and
sutures joined to the end portion and extending inside the shaft portion;
wherein the length of the shaft portion may be adjusted after the implanting of the bone anchor assembly into the bone by removing shaft segments.

2. The bone anchor assembly of claim 1, wherein the shaft segments have tabs at one axial end and blanks at the opposite axial end so that the shaft segments interlock by mating the tabs of one shaft segment with the blanks of an adjacent shaft segment.

3. The bone anchor assembly of claim 2, wherein the tabs have grooves extending in the axial direction.

4. The bone anchor assembly of claim 1 further comprising:
a collar configured to slide along the shaft portion.

5. The bone anchor assembly of claim 4, wherein the shaft segments have radial holes and the collar has at least one radial hole enabling viewing of alignment of the at least one radial hole of the collar with a radial hole of a shaft segment.

6. The bone anchor assembly of claim 4, wherein the collar has inwardly movable elements.

7. The bone anchor assembly of claim 6, wherein the inwardly movable elements are sliding pistons.

8. The bone anchor assembly of claim 7, wherein the pistons are biased so as not to contact the shaft segments, and forcing the pistons to slide against the biasing moves the tabs of a shaft segment out of engagement with the blanks of an adjacent shaft segment.

9. The bone anchor assembly of any of claim 1 further comprising:
a hollow driving rod sized and shaped to fit into the shaft portion and to engage the end portion for rotation;
wherein, upon engagement of the driving rod with the end portion, the sutures extend inside the driving rod.

10. The bone anchor assembly of claim 9, wherein the driving rod has an engagement portion with a non-circular cross-section for engagement with the end portion.

11. The bone anchor assembly of claim 1:
wherein the elongated hollow shaft portion extends along a shaft axis; and
wherein shaft segments are removed by applying a force to the shaft portion in a direction normal to the shaft axis and sufficient to deform the shaft portion to cause the shaft portion to break at one of the shaft segment boundaries between two adjacent shaft segments.

12. The bone anchor assembly of claim 11, wherein the shaft segments have external cylindrical walls having a first diameter, and the shaft segment boundaries have external cylindrical walls having a second diameter that is less than the first diameter.
